(19) United States
(12) Patent Application Publication
(10) Pub. No.: US 2015/0099924 A1
(43) Pub. Date: Apr. 9, 2015

(54) METHODS AND DEVICES FOR REPAIR OF VAGINAL WALL OR UTERUS

(71) Applicant: CAREY TASCA PTY LTD, Eltham (AU)

(72) Inventor: Marcus Patrick Carey, Eltham (AU)

(73) Assignee: Carey Tasca Pty Ltd, Eltham (AU)

(21) Appl. No.: 14/396,997

(22) PCT Filed: Apr. 24, 2013
(86) PCT No.: PCT/AU2013/000415

§ 371 Date: Oct. 24, 2014

Related U.S. Application Data
(60) Provisional application No. 61/639,621, filed on Apr. 27, 2012.

(51) Int. Cl. A61F 6/12; A61F 2/00

(52) U.S. Cl. CPC A61F 6/12; A61F 2/0063; A61F 2002/0072; A61F 2220/0016; A61F 2250/006; A61F 2220/0033; A61F 2250/0059
USPC 600/37

(57) ABSTRACT

A splinting appliance for use in repair of the vaginal wall or uterus includes a body portion, securing means and coupling means. The body portion is shaped to support a substantially normal vaginal apex. The securing means is configured to enable removable securing of the splinting appliance within the vaginal canal. The coupling means enables the splinting appliance to releasably couple with a vaginal elevator device when inserted in the vaginal canal during surgery. Use of the vaginal elevator device when coupled with the splinting appliance aids in location of the vaginal apex. The splinting appliance provides a substantially rigid support which, when in use, facilitates manipulation of tissue at the vaginal wall when approaching through the abdomen e.g. for dissection and/or attachment of a repair graft.

### FIELD OF THE INVENTION

This invention relates to a method for surgical repair of a vaginal wall or uterus due to pelvic organ prolapse. It relates particularly but not exclusively to a method performed by laparoscopy or other minimally invasive technique which provides abdominal access to the vaginal wall, and a splinting apparatus for use during the surgery and during initial convalescence of the woman undergoing treatment and elevator device for use during surgery.

### BACKGROUND TO THE INVENTION

Vaginal prolapse is a condition in which the bladder, uterus and/or bowel protrude into the vagina, typically due to loss of natural support for the pelvic organs and the vaginal vault in women who have undergone a prior hysterectomy. In the normal female anatomy, direct support for the vaginal vault is provided by the parametrium (cardinal and uterosacral ligaments) and paracolpium fibers. These fibers act like suspensory ligaments and arise from the fascia of the piriformis muscle, sacroiliac joint and lateral sacrum, and insert into the lateral upper third of the vagina. Indirect support for the vaginal vault is provided by the levator plate, formed by the fusion of the right and left levator ani muscles between the rectum and coccyx. Pelvic organ prolapse and vaginal vault prolapse occurs after failure of these direct and indirect supporting mechanisms and is frequently accompanied by weakness of the muscular pelvic floor and suspensory fibers of the parametrium and upper paracolpium. Vaginal vault prolapse occurs in approximately 10% of women following hysterectomy and occurs in equal numbers following abdominal and vaginal hysterectomy.

Traditional surgery for repair of pelvic organ prolapse involves making incisions in the vaginal wall and strengthening the fascial tissues with stitches to reinforce the repair. This vaginal repair approach has a high failure rate with around 30% of women surgically treated for prolapse requiring repeat surgery for recurrent pelvic floor symptom. Women aged less than 60 years and women with stages 3 and 4 prolapse are more likely to experience recurrent prolapse after vaginal repair.

Abdominal sacral colpopexy (ASC) is a procedure that was developed to address the high rate of failure with vaginal surgery to treat vaginal vault prolapse. ASC employs retroperitoneal interposition of a suspensory prosthesis (typically a mesh) between the vaginal apex and the sacrum. In addition to the high success rate reported with the ACS procedure, other advantages include preservation of vaginal capacity resulting in maintenance of coital function. Significant problems associated with the ASC operation include de novo postoperative stress incontinence, intraoperative hemorrhage, dyspareunia and vaginal mesh exposure. Use of a laparotomy incision brings with it risk of infection and long recovery time.

The use of laparoscopy to treat pelvic organ prolapse has steadily increased over the past 30 years. Laparoscopic sacrocolpopexy (LSC) is performed in an identical manner to ASC with the only difference being the smaller laparoscopic incisions which avoid a major laparotomy incision. Thus, LSC is associated with less postoperative pain, shorter hospital stay, and a quicker return to normal activities. However, current methods of LSC require a high level of laparoscopic surgical skill and expertise. Thus, use of laparoscopy for the treatment of pelvic organ prolapse has been limited to a relatively small number of surgeons with sufficient laparoscopic and reconstructive pelvic surgical skills to perform these procedures safely and effectively.

It would be desirable to provide an alternative method and related devices for performing vaginal wall repair which overcomes or at least ameliorates one or more of the disadvantages of the prior art.

The discussion of the background to the invention included herein including reference to documents, acts, materials, devices, articles and the like is intended to explain the context of the present invention. This is not to be taken as an admission or a suggestion that any of the material referred to was published, known or part of the common general knowledge as at the priority date of any of the claims.

### SUMMARY OF THE INVENTION

According to one aspect of the present invention, there is provided a splinting appliance for use in repair of the vaginal wall or uterus, the appliance including:

a body portion shaped to support a substantially normal vaginal apex;

securing means configured to enable removable securing of the splinting appliance within the vaginal canal; and

coupling means for removably coupling the splinting appliance with a vaginal elevator device when inserted in the vaginal canal during surgery, wherein use of the vaginal elevator device coupled with the splinting appliance aids in location of the vaginal apex;

wherein the splinting appliance provides a substantially rigid or semi-rigid support which, when in use, facilitates manipulation of tissue at the vaginal wall when approaching through the abdomen.

The splinting appliance is intended for use with aspects of the invention relating to a method for repairing a vaginal wall or uterus damaged by pelvic organ prolapse. Thus, use of the splinting appliance facilitates dissection of tissue from the vaginal wall and attachment of a repair graft to the vaginal wall. Such method is outlined in further detail below. In this description of the invention including the claims, the term “vaginal wall” is intended to include either the anterior vaginal wall or the posterior vaginal wall, or both the anterior vaginal wall and posterior vaginal wall, and includes the vaginal apex (vaginal vault), or part of any of the foregoing.

Preferably, the splinting appliance coupling means is configured to permit decoupling and removal of the vaginal elevator device from the splinting appliance with minimal retraction of the splinting appliance within the vaginal canal. This ensures that optimal placement of the splinting appliance relative to the reinforcing implant is substantially maintained throughout the critical stages of the repair including suturing and tissue in-growth. The coupling means may include one or more hollow portions for receiving correspondingly shaped portions of the vaginal elevator device. Alternatively, the coupling means may include a recess configured to cooperate with a protrusion on a shaft of an elevator device to engage and controllably manipulate and elevate the splinting appliance. In another embodiment, the coupling means may have one or more protrusions adapted to cooperate with one or more recesses toward the distal end of the elevator device, for releasable engagement with the elevator. Ideally, these components of the coupling means have minimal frictional fit so that there is little, if any movement of the splinting appliance during de-coupling of the vaginal elevator device.

The vaginal splinting appliance particularly the body portion, is preferably manufactured from a somewhat-rigid, biocompatible and non-reactive material such as medical grade silicon or polyvinyl chloride (PVC) or other suitable polymer. The external surface of the body portion may have a low coefficient of friction. To minimize the risk of infection, the appliance may also be coated or impregnated at least in part, with an antimicrobial composition such as triclosan, silver or the like. Where the body portion is manufactured from a harder material such as Hytrel, it may be desirable provide an external coating of a softer material such as e.g. silicon, polyurethane medical film, polyether or thermoplastic polyurethane (e.g. Texin® or Desmopan®) or other moldable and/or soft and/or atraumatic material to minimize patient discomfort. In some embodiments, the splinting appliance may be entirely or partly manufactured from such materials which, when there is sufficient thickness in the body portion 501, provides rigid support for dissection and suturing (as well as splinting) without compromising patient comfort during placement and removal.

The splinting appliance need not be provided with solid wall portions. It is to be understood that there may be openings/voids in the anterior and/or posterior surfaces of the body portion to reduce material volume. Ideally wall portions are provided in the regions required to support manipulation of tissue. The somewhat rigid wall portions facilitate e.g. dissection of tissue from the vaginal wall, while openings/voids or channels may further act as a guide to indicate where tissue should remain intact.

In one embodiment, the securing means includes one or more inwardly directable extension arms (i.e. arms that may be directed in toward each other) configured to be removably attached to the vaginal wall. Attachment may be facilitated by suture eyelets formed in the extension arms through which a single suture may be placed to secure the splinting appliance in the desired position in the vaginal canal. However hooks, barbs, tacks and other removable fasteners are also contemplated.

In one embodiment, the securing means includes two or more extension arms configured to be joined to one another at one or more positions along the respective extension arms. Adjustability in the location of the join between the extension arms permits variable sizing, effectively modifying the overall length of the device according to the position of the join, to suit the recipient. Further, with the arms joined a ‘belt’ is formed which enables easy removal of the splinting appliance post-operatively by grasping or hooking onto the ‘belt’ and withdrawing the splinting appliance from the vaginal canal after the securing means have been removed or disabled.

In an embodiment, the extension arms include portions that separable from the appliance to modify its overall length. These may be trimmable or tearable optionally with weakened portions such as perforations or a frangible line to assist tearing off. Alternatively/additionally the body portion may include one or more removable sections adapted to shorten the body portion when removed. These removable sections are ideally provided toward the apical end of the body portion and enable the body portion to be shortened to match the anatomy of the recipient.

According to another aspect of the present invention, there is provided a method for repairing a vaginal wall damaged by pelvic organ prolapse in a subject, including the steps of:
(a) forming an access opening to provide abdominal access to the vaginal apex;
(b) locating a splinting appliance in the vaginal canal;
(c) introducing a reinforcing implant, having a body and a tongue, through the access opening and positioning the body of the reinforcing implant over a portion of the vaginal wall for repair;
(d) with the splinting appliance positioned in the vaginal canal, attaching at least a portion of the body of the reinforcing implant to the vaginal wall;
(e) positioning the tongue of the reinforcing implant anterior to the sacrum; and
(f) securing the splinting appliance in the vaginal canal for a duration sufficient to achieve tissue in-growth into the reinforcing implant;

wherein securing the splinting appliance in the vaginal canal substantially stabilizes placement of the reinforcing implant while tissue in-growth is occurring.

According to yet another aspect of the present invention, there is provided a method for repairing a uterus damaged by pelvic organ prolapse in a subject, including the steps of:
(a) forming an access opening to provide abdominal access to the uterus;
(b) locating a splinting appliance in the vaginal canal;
(c) introducing a reinforcing implant, having a body and a tongue, through the access opening and positioning the body of the reinforcing implant over a portion of the uterus for repair;
(d) with the splinting appliance positioned in the vaginal canal, attaching at least a portion of the body of the reinforcing implant to one or both of the uterus and the vaginal wall;
(e) positioning the tongue of the reinforcing implant anterior to the sacrum; and
(f) securing the splinting appliance in the vaginal canal for a duration sufficient to achieve tissue in-growth into the reinforcing implant;

wherein securing the splinting appliance in the vaginal canal substantially stabilizes placement of the reinforcing implant while tissue in-growth is occurring.

Preferably, securing the splinting appliance in the vaginal canal includes removably attaching at least part of the splinting appliance to the vaginal wall to restrict movement of the splinting appliance during the critical stages of repair and tissue in-growth. The splinting appliance is removed when tissue in-growth has occurred. This may be achieved in 20 to 30 days although it is typical for satisfactory in-growth to have occurred within 22 to 27 days of implantation of the reinforcing implant. Most desirably, the vaginal splinting appliance is removed about 25 days after implantation where, in most cases, tissue in-growth sufficient to resist reinforcing implant pull-out has been achieved.

Ideally, prior to attaching the body of reinforcing implant to the vaginal wall, a vaginal elevator device is coupled with the splinting appliance and used to elevate the vaginal vault to facilitate location of the vaginal apex prior to attaching the reinforcing implant to the vaginal/uterus wall. The elevator device is de-couplable from the splinting appliance with minimal retraction of the splinting appliance.

Thus, viewed from another aspect, the present invention provides a vaginal elevator device for use with a vaginal splinting appliance, the elevator device comprising:
(a) coupling zone configured for releasable coupling with the vaginal splinting appliance;
(b) a shaft attached to the coupling zone; and
(c) a handle portion on the shaft for gripping by a user.

Preferably, the coupling zone is configured to decouple from the splinting appliance, when in use, with minimal retraction of the splinting appliance from the vaginal canal. This enables the optimal position of the splinting appliance relative to the implant and the vaginal apex/walls to be maintained. The coupling zone may include a protrusion configured to couple within a cooperatively shaped recess formed in the splinting appliance to engage and controllably manipulate the splinting appliance using the vaginal elevator. Alternatively, the coupling zone may include a larger body portion adapted to fit within a hollow portion of a vaginal splinting device. Typically, the handle portion and the shaft are oriented at approximately 30 degrees to one another.

In a preferred embodiment, the reinforcing implant is a synthetic bio-compatible implant having a braided, porous, laser-cut, woven and/or mesh-like construction with a plurality of openings facilitating tissue in-growth. The openings may be pits formed in the surfaces of the body and/or tongue of the reinforcing implant into which tissue in-growth is desirable, or the openings may be through-holes. Preferably the reinforcing implant has a periphery which is laser cut.

Preferably, there is substantially tensionless placement of the reinforcing implant prior to tissue in-growth. That is, the reinforcing implant is not in tension immediately following its positioning. It is desirable to avoid use of sutures, anchors, clips, staples or other fasteners to maintain the position of the tongue of the reinforcing implant anterior to or in contact with the sacrum. Instead, it is desirable to stabilize placement of the reinforcing implant using the vaginal splinting appliance which remains in the vaginal canal for a duration sufficient to achieve tissue in-growth into the reinforcing implant. In one embodiment, ideal placement is with the tongue of the reinforcing implant resting along the anterior curvature of the sacral hollow and/or sacral promontory. A biocompatible resorbable adhesive may be used to assist with and/or stabilize placement prior to tissue in-growth.

In one embodiment, the method includes, prior to attaching the body of reinforcing implant to the vaginal wall, elevating the vaginal vault to facilitate location of the vaginal apex. This assists with positioning the reinforcing implant prior to attaching at least a portion of the body of the reinforcing implant to the vaginal wall. Vaginal wall elevation may be achieved using a vaginal elevator device when coupled with the splinting appliance and inserted into the vaginal canal.

In one embodiment, the method includes forming an opening in the peritoneum anterior to the sacral promontory, and locating a portion of the implant tongue within the opening. The opening may be an incision or a cavity formed in the pre-sacral space between the peritoneum and the sacral promontory. The end of the tongue of the reinforcing implant may include a widened portion, a barb, lateral projection or other feature which substantially limits retraction of the tongue from the opening. Alternatively, the tongue of the reinforcing implant may simply rest in contact with the peritoneum of the sacral hollow and/or sacral promontory and become fixed in that position after tissue in-growth has occurred. This latter approach avoids forming an opening in the peritoneum.

Where a peritoneal opening is used, it is preferred that the opening is formed by creating a first peritoneal opening anterior to the sacral promontory; and forming a tunnel from the first peritoneal opening into the pre-sacral space, wherein the tunnel receives at least a portion of the tongue of the reinforcing implant in the region of the sacral curve. Forming the opening may further include creating a second peritoneal opening, preferably below the first peritoneal opening, and extending the tunnel to the second peritoneal opening. Thus, the tunnel is formed between the peritoneal openings, in the pre-sacral space between the peritoneum and the sacrum. Preferably, each peritoneal opening is a transverse incision in the midline allowing entry into the pre-sacral space. The transverse incisions may have a length of 1 to 3 cm, preferably 1.5 to 2.5 cm and more preferably 1.5 to 2 cm.

In one embodiment, the tongue of the reinforcing implant is drawn through the tunnel until at least a portion of the tongue of the reinforcing implant rests superior to the peritoneal openings and in abutment with the anterior longitudinal ligament.

The method may be used to repair the anterior, posterior or both anterior and posterior vaginal walls. The method may also be used to repair the vaginal apex (vaginal vault). The method may also be used to repair the vaginal apex (vaginal vault) after concomitant hysterectomy or sub-total hysterectomy. The method may be used to repair prolapse of the uterus in women wishing to retain their uterus.

When the vaginal wall being repaired is the posterior vaginal wall, with the splinting appliance positioned in the vaginal canal, a posterior arm of the body of the reinforcing implant is positioned onto an upper portion of the posterior vaginal wall and is attached to the vaginal wall, preferably by tacking with a single resorbable suture placed laparoscopically. When the vaginal wall being repaired is the anterior vaginal wall, with the splinting appliance positioned in the vaginal canal, an anterior arm of the body of the reinforcing implant is positioned onto an upper portion of the anterior vaginal wall and is attached to the vaginal wall, preferably using a single resorbable suture placed laparoscopically.

When the prolapsed uterus is being repaired, with the splinting appliance positioned in the vaginal canal, a posterior arm of the body of the reinforcing implant is positioned onto an upper portion of the posterior vaginal wall and in contact with the supra-vaginal portion of the posterior cervix and is attached to the posterior cervix at this level, preferably by tacking with a single resorbable suture placed laparoscopically. However, in some surgeries it may be desirable to attach an arm of the body of the reinforcing implant to part of the uterus only, typically the lower portion of the uterus, without attachment to the vaginal wall.

Whether the repair is to the vaginal wall (or apex) or the uterus, the single suture attaching the anterior or posterior arm of the body of the reinforcing implant may be replaced with one or more staples, clips, tacks or fasteners placed abdominally, e.g. using laparoscopic techniques, facilitated by placement of the splinting appliance in the vaginal canal to provide a support surface on which to create the suture and place the suture, staple, clip etc.

Preferably, the method is performed using minimally invasive surgical techniques, such as by laparoscopy. The method may be performed, at least in part using robotic surgical techniques or by laparotomy. Forming the access opening may further involve separating at least a portion of the posterior and/or anterior vaginal wall from surrounding pelvic organ tissue to facilitate positioning the body of the reinforcing implant on the anterior and/or posterior vaginal wall. Dissection of this tissue is also facilitated by placement of the splinting appliance in the vaginal canal.

In women with the uterus present, concomitant hysterectomy or sub-total hysterectomy may be performed prior to the steps of the inventive method. In women with the uterus present and wanting preservation of the uterus, forming the access opening may further involve separating at least a portion of the posterior vaginal wall from surrounding pelvic organ tissue to facilitate positioning the body of the reinforcing implant on the posterior vaginal wall and posterior cervix. Again, placement of the splinting appliance in the vaginal canal assists with this dissection process by providing a support for the vaginal wall during tissue lifting and cutting.

In accordance with another aspect of the present invention, there is provided a software program product embodying instructions for controlling a robotic surgical apparatus performing steps in a method described in the foregoing.

In accordance with another aspect of the present invention still, there is provided a software program product embodying instructions for providing an interface between a user and a robotic surgery control system with which a user plans or executes a robotic surgery according to a method described in the foregoing.

Aspects of the invention may be incorporated into a kit or packaged product including one or more reinforcing implants, one or more vaginal splinting appliances of e.g. varying sizes or a splinting appliance having removable portions that permit re-sizing, and instructions for performing a surgical method for repairing a vaginal or uterine wall damaged by pelvic organ prolapse, as described herein.

The instructions may be provided in written form, and/or in pictographic form. Alternatively/additionally, the instructions may be provided in the form of audio and/or video content in which the method is demonstrated with a real example or pictographic example, and recorded in an electronic format such as mp3 or avi file and stored on DVD or other electronic storage media. The kit may also include a vaginal elevator device configured for releasable coupling with the vaginal splinting appliance.

Thus, the invention is intended to cover methods, splinting appliances, elevators and kits for use with a reinforcing implant used to repair the prolapse defect by way of tissue on-growth, as well as or as an alternative to tissue in-growth.

### BRIEF DESCRIPTION OF THE DRAWINGS

The invention will now be described with reference to the accompanying drawings in which some preferred embodiments are illustrated. In the drawings:

FIG. 1 is a schematic representation showing the vaginal canal anteriorly in the vicinity of the sacrum, and showing a portion of the sacral peritoneum, with a vaginal splinting appliance prior to insertion into the vaginal canal.

FIG. 2 is schematic representation similar to FIG. 1, showing a vaginal splinting appliance located in the vaginal canal with extension arms joined together and to the vaginal wall, and anterior and posterior arms of a reinforcing implant attached to the anterior and posterior vaginal walls, with a tongue of the implant passed through a tunnel formed in the peritoneum.

FIGS. 3a and 3b are schematic representations showing front and side views respectively of a vaginal splinting appliance according to an embodiment of the invention.

FIGS. 4a and 4b are schematic representations showing front and side views respectively of a vaginal elevator device for use with an embodiment of the invention.

FIG. 5a is a schematic representation showing the vaginal elevator device of FIGS. 4a and 4b coupled with the splinting appliance of FIGS. 3a and 3b. FIG. 5b is a schematic representation of the vaginal elevator device decoupled from the splinting appliance and being removed. FIG. 5c is a schematic representation showing the splinting appliance with extension arms joined.

FIGS. 6a, 6b and 6c are front, side and perspective views of a reinforcing implant which may be used during repair of the vaginal wall using embodiments of the invention.

FIGS. 7a to 7d are schematic representations showing alternative embodiments for the splinting appliance and elevator device and showing an alternative coupling between these parts.

### DETAILED DESCRIPTION

The present invention provides improvements in a vaginal splinting appliance, elevator device and method for repair of a vaginal wall or uterus as described in

International patent application PCT/AU2011/001385 filed 28 Oct. 2011, the entire contents of which are hereby incorporated herein by reference.

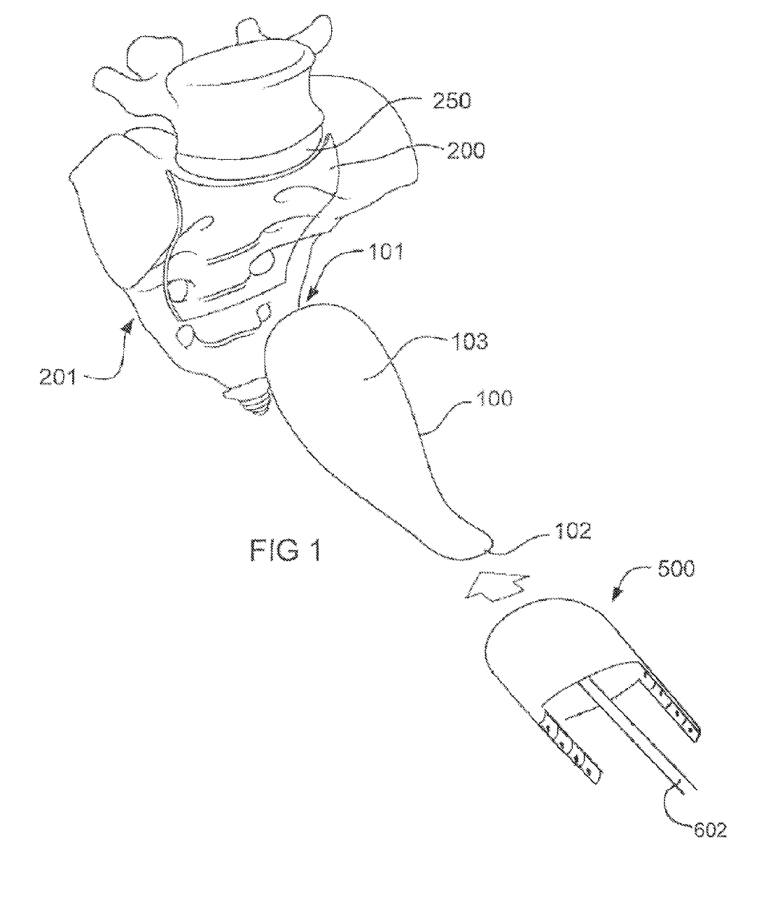

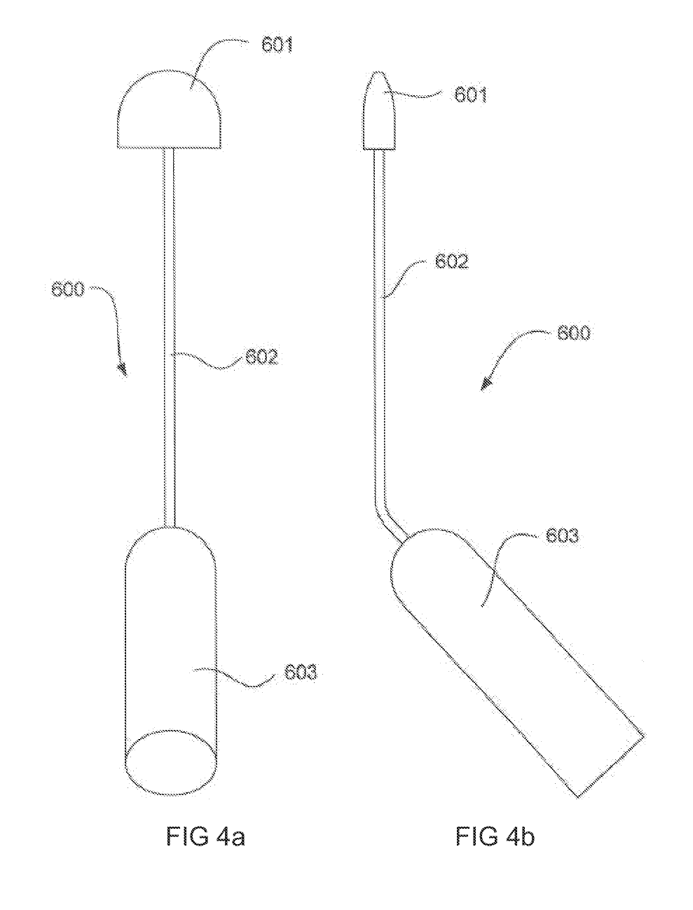

Referring firstly to FIG. 1, components of the pelvic anatomy of a woman are shown in a schematic illustration. Vaginal canal 100 has apex 101 and opening 102 into which vaginal splinting appliance 500 is inserted. Prior to insertion, the vaginal splinting appliance is coupled with the body 601 of a vaginal elevator device 600 (see FIGS. 4a, 4b) of which only shaft 602 is visible in FIG. 1. A portion of the sacral peritoneum 200 is shown, overlaying part of the sacrum 201 in the region of the sacral curve.

Following induction with general anesthesia the woman is placed in a low lithotomy position. Preferably the inventive surgical method is performed via laparoscopy or other minimally invasive technique although it may also be performed via a laparotomy incision or by robotic or robotic-assisted surgery through the abdomen.

Standard techniques are used to introduce a 10 mm operating laparoscope at the umbilicus. Under laparoscopic vision, two ports are inserted through the anterior abdominal wall into the peritoneal cavity. For a right-handed surgeon this includes a 10 mm port on the patient's left and a 5 mm port on the patient's right. Preferably these ports are sited two fingerbreadths above and two fingerbreadths medial to the anterior superior iliac spine to avoid injury to the inferior epigastric artery and vein and damage to the lateral cutaneous nerve to the thigh. A further 5 mm port is introduced suprapubically in the midline under laparoscopic vision.

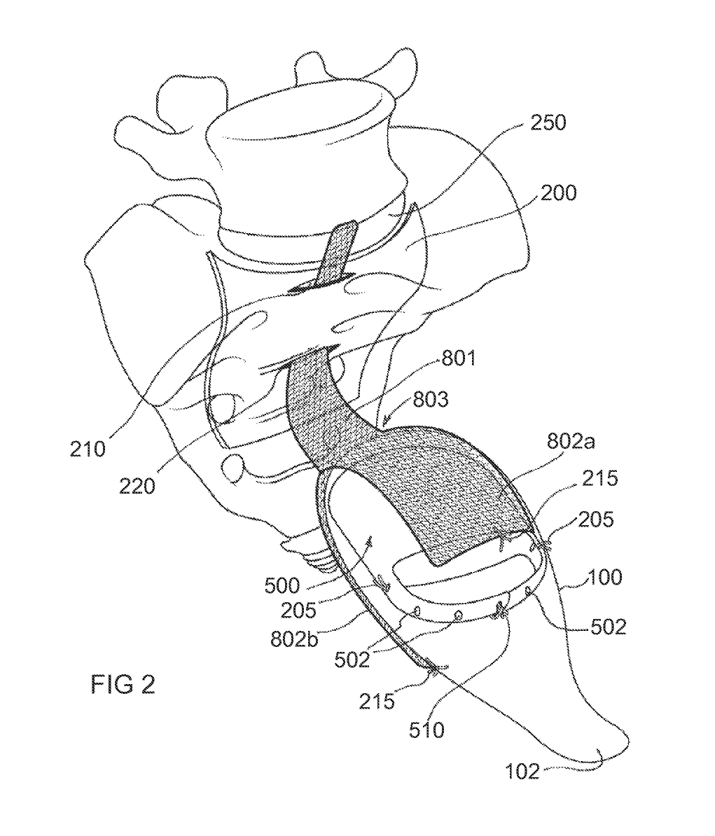

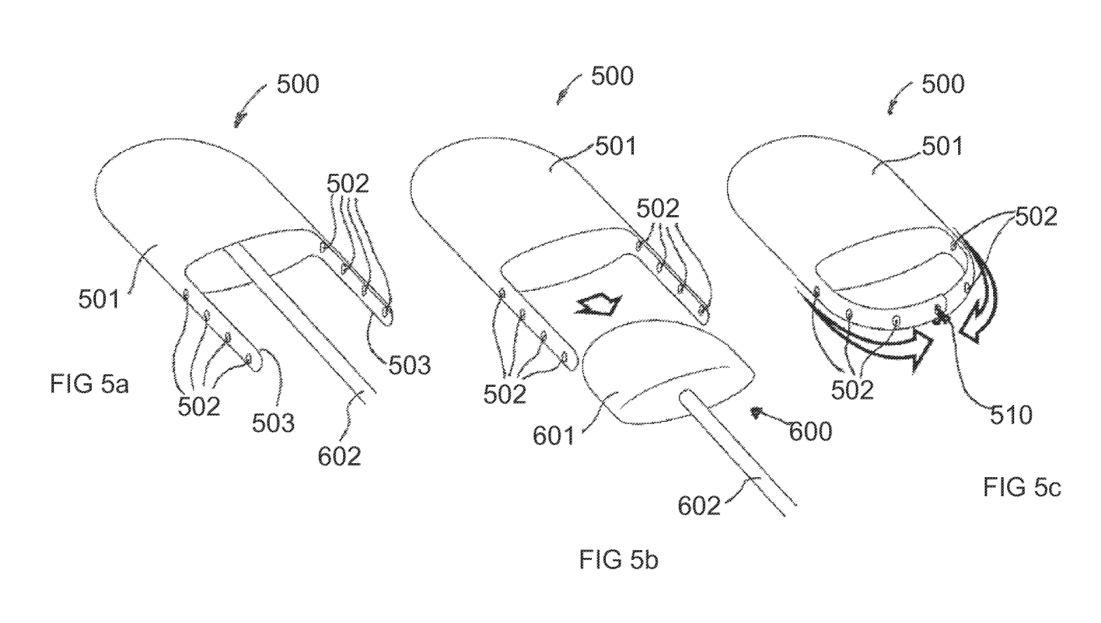

The vaginal splinting appliance 500 is inserted into the vagina 100 as is illustrated in FIG. 2 aided by a vaginal elevator device 600 (FIGS. 4a, 4b, 5a, 5b, 7a-7d). FIG. 5a shows the vaginal elevator device 600 coupled with splinting appliance 500. Once in position, the splinting appliance 500 is sutured to the vaginal walls using sutures 205, with one suture located in each of the lateral vaginal walls at a level above the hymen remnant. Preferably, sutures 205 are made through suture eyelets 502 of the splinting appliance although other attachment means such as staples and clips are contemplated. A single suture through only one extension arm 503 is also contemplated.

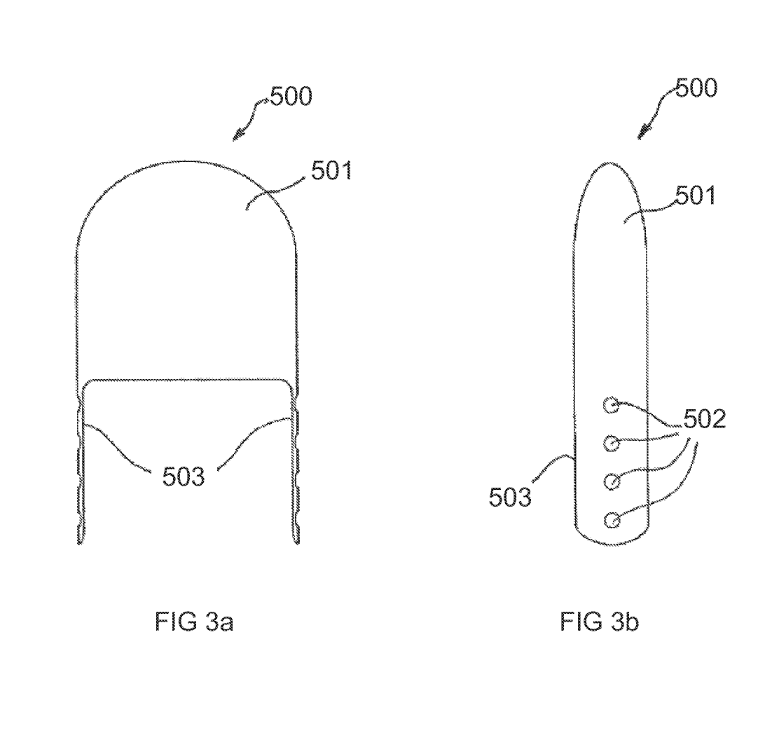

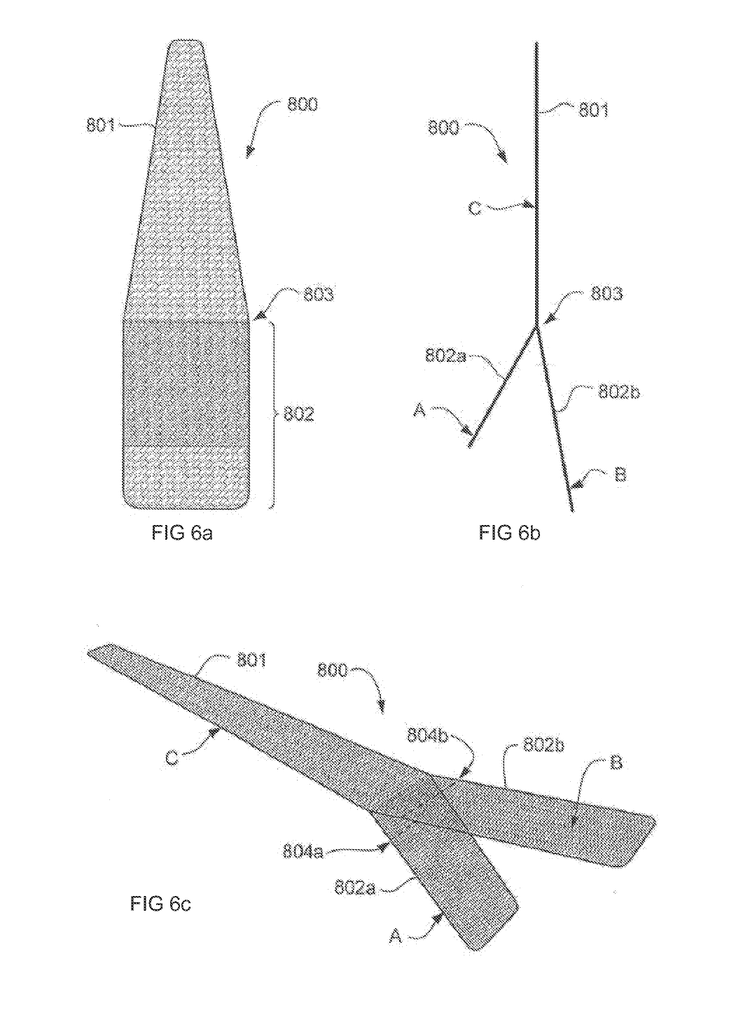

FIGS. 3a and 3b illustrate front and side views respectively of a vaginal splinting appliance according to an embodiment of the present invention. The vaginal splinting appliance may be manufactured from semi-rigid biocompatible, non-reactive material (e.g. medical grade silicon, PVC) with a relatively smooth external surface. In an embodiment, at least a body portion 501 of the splinting appliance is formed from medical grade silicon or a relatively rigid material such as e.g. Hytrel. The appliance provides a somewhat rigid smooth support during dissection of tissue from the vaginal walls and during suturing of a graft material (such as the mesh implant in FIGS. 2 and 6a to 6c) to the vaginal wall. These benefits are realized by placement of the splinting appliance prior to dissection and suturing, and are in addition to the benefit of the “splinting” effect of the appliance after surgery, which aids in maintaining optimal positioning of the graft relative to the vagina during tissue in-growth, even during patient movement post-surgically.

The splinting appliance is secured in position for a period of three to four weeks while there is tissue in-growth. To minimize the risk of infection occurring, the appliance may also be coated or impregnated at least on an external surface with an antimicrobial composition such as triclosan, silver or the like. To minimize discomfort to the patient arising from hardness of the body portion, the splinting appliance may be provided with an external coating of a softer material such as e.g. silicon, polyurethane medical film, polyether or thermoplastic polyurethane (e.g. Texin® or Desmopan®) or other moldable and/or soft and/or atraumatic material. In some embodiments, the splinting appliance may be entirely or partly manufactured from such materials which, when there is sufficient thickness in the body portion 501, provides rigid support for dissection and suturing (as well as splinting) without compromising patient comfort during placement and removal.

The splinting appliance includes a body portion 501 and extension arms 503 with attachment means. In the embodiment illustrated, the attachment means are in the form of 4 pairs of suture eyelets 502 provided on extension arms 503. The extension arms may be trimmed for re-sizing although that need not be the case. Alternatively/additionally, the body portion may be modified to incorporate trimmable or frangible sections which can be removed or cut down to shorten the body portion of the splitting appliance from the apical end.

In one embodiment, once the splinting appliance has been placed in the vaginal canal the extension arms may be joined using eyelet pairs 502 as shown in FIGS. 2 and 5c. The length of the splinting appliance may be adjusted like a belt, by appropriate selection of the suture eyelets to be joined, based on the woman's anatomy. The eyelet pairs selected for joining need not be symmetrical.

The extension arms may be trimmed, e.g., to avoid or limit the amount of overlap in extension arms which are joined. Thus the suture eyelets in the extension arms may be multi-purpose, enabling the splinting appliance to be secured in the vaginal canal, as well as permitting resizing of the appliance by joining the extension arms at a desired location along the extension arm length. Typically, the extension arms are joined using a single suture. However, it is contemplated that any suitable attachment means may be employed such as a toothed join, adhesive, tack, hook and eye, cable tie join, snap closure, click-fit frictional join or the like. Alternatively, where the extension arms are sufficiently flexible they may be tied or knotted together. Trimming may occur before or after joining the extension arms if trimming is necessary or desirable e.g., to avoid or limit the amount of overlap in extension arms which are joined. Ideally, the extension arms are joined after the splinting appliance has been secured in the vaginal canal (e.g. using sutures 205) although it is contemplated that the splinting appliance may be sized by joining the extension arms prior to placement within the vaginal canal in some circumstances.

Preferably the extension arms 503 are directed inwardly to minimize discomfort when the appliance is positioned in the vagina and during removal after tissue in-growth has occurred. Inward direction of the extension arms may be achieved by joining the extension arms at join 510. Alternatively/additionally, the splinting appliance 500 may be manufactured with the extension arms 503 directed slightly inwardly e.g. at an angle of 1 to 8 degrees, preferably 3 to 6 degrees directed inwards from square with the body portion 501. It is preferable in some embodiments that the extension arms are not too rigid and may, therefore be likened to straps. Although the extension arms 503 shown the drawings appear curved consistently with the convex shape of the body portion sidewalls, that need not be the case and they may be completely or substantially flat.

In the embodiments illustrated, the body portion 501 has a double convex profile, with convex external shaping provided in both front and side orientations as seen in FIGS. 3a and 3b. This provides strength while also substantially matching the geometry of the vagina and so minimizing discomfort when in use. This shaping is also advantageous because it provides a broad support surface during dissection of tissue from the vaginal wall when preparing the site for attachment of the repair graft/implant.

Various sizes and dimensions are contemplated for the vaginal splinting appliance 500. In one embodiment, the splinting appliance is approximately 8.5 cm in overall length and approximately 4 cm in overall width. In such embodiment, body 501 is approximately 5 cm long and approximately 4 cm wide with a maximum front-to-back thickness of approximately 1.25 cm. In an embodiment, body portion 501 has a hollow interior for receiving the body 601 of a vaginal elevator device 600 although other coupling means are contemplated and described below. In one embodiment, extension arms 503 are approximately 3 cm long and 1 cm wide and may be frangible such that the extension arms may be shortened by trimming or tearing off part of the extension. This enables the splinting appliance to be appropriately sized for an individual patient.

According to the inventive method, the splinting appliance is placed in the vagina prior to dissection of tissue from the vaginal wall and preferably, prior to the commencement of the surgical part of the repair procedure. The splinting appliance remains in place, secured by sutures or the like for the duration of the procedure including suturing of the repair graft/implant to the vaginal walls and the support provided by the splinting appliance 500 is maintained until tissue in-growth into the reinforcing implant fixes the implant in position. This helps to provide substantially tensionless placement of the reinforcing implant.

Because the splinting apparatus is placed in the vagina before placement of the repair graft/implant and is not removed until tissue in-growth has occurred, it enables optimal contact to be maintained between the graft/implant and the vagina consistently, throughout the critical stages of the repair. This provides a significant difference over prior art approaches which require exchange of devices (removal of elevator and insertion of splint) at the conclusion of surgery, which can lead to movement of the graft/implant and discomfort for the patient. Optimal placement of the repair graft/implant at the sacral promontory seen at the vaginal apex can be confirmed by MRI.

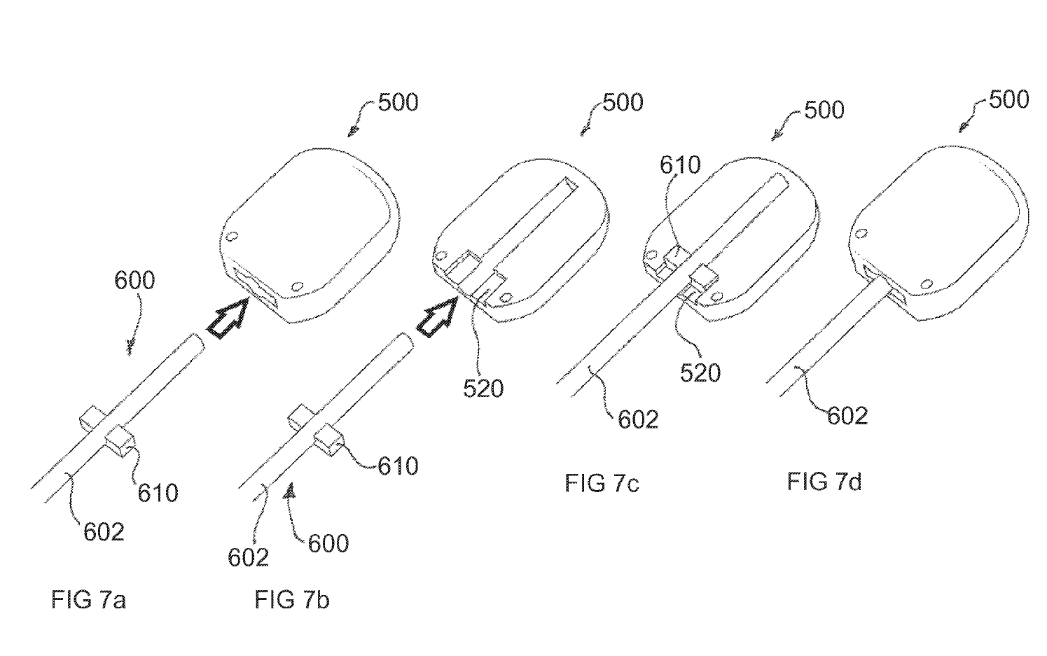

Now turning to FIGS. 4a and 4b, front and side views of a vaginal elevator device 600 are shown. Vaginal elevator device includes a body portion 601, a shaft 602 and a handle portion 603. The body portion is shaped to fit inside a hollow formed in the splinting appliance 500 during surgery as shown in FIGS. 5a and 5b although other coupling arrangements may be contemplated, such as that shown in FIGS. 7a to 7d. In the embodiment illustrated in FIGS. 4a and 4b, the elevator device body portion 601 is approximately 3 cm wide, 3 cm long and with thickness that can be accommodated within a hollow in the vaginal splinting appliance (e.g. 1 cm). The shaft 602 is approximately 15 cm long and attaches at approximately 30 degrees to handle 603 which is approximately 12 cm in length. The vaginal elevator device 600 facilitates location of the vaginal apex and thereby assists with optimal placement of the implant onto the vaginal walls during the inventive surgical method. Ideally, the splinting appliance and the elevator device are readily decouplable without displacing or retracting the splinting appliance. In this way a slight friction fit may be acceptable but this fit should not be so strong that retraction of the splinting appliance occurs when decoupling the elevator device prior to removal from the vaginal canal.

An alternative embodiment of an elevator device 600 and splinting appliance 500 is shown in FIGS. 7a to 7d. FIGS. 7b and 7c show the splinting appliance 500 in cross section to illustrate more clearly the alternative coupling arrangement. Here, the coupling zone comprises a protrusion in the form of a cross bar 610 on shaft 602. The cross bar is configured to be releasably coupled with the splinting appliance at recess 520. The crossbar 610, when coupled with recess 520, permits the user to engage and controllably manipulate the splinting appliance 500 to locate the vaginal apex, whilst minimizing the frictional connection between the two parts. Decoupling the elevator device 600 from the splinting appliance 500 requires simple retraction of the cross bar 610 from the recess 520 and avoids retraction or movement of the splinting appliance from the optimal position when placed within the vaginal canal.

The vaginal elevator device 600 is inserted inside the vaginal splinting appliance 500 for placement and to assist the surgeon to identify the vaginal apex (vaginal vault). A suture is placed through a suture eyelet 502 on at least one extension arm 503 to secure the position of the splinting appliance in the vagina. With the vagina elevated cranially using the elevator with body portion 601 coupled with the splinting appliance 500, the surgeon opens the peritoneum transversely at the vaginal apex using a dissection technique. This is facilitated by the substantially smooth and rigid or semi-rigid surface of the splinting appliance supporting the vaginal wall, which provides the surgeon with resistance to accurately perform dissection. Dissection continues posteriorly into the rectovaginal septum so that the peritoneum is dissected off the upper half of the posterior vaginal wall. At around this point the rectovaginal septum is entered and the rectum is dissected off the middle and lower posterior vaginal wall. On occasion, dissection can be further facilitated by using a rectal probe in addition to the splinting appliance. This dissection is carried out in the midline and extended laterally on both sides to the insertion of the uterosacral ligaments.

The bladder is then dissected off the upper anterior vaginal wall. This dissection continues to approximately the midpoint of the anterior vaginal wall. Dissection is continued laterally towards both bladder pillars but generally dissection of the bladder pillars is avoided. This is to reduce intra-operative bleeding and injury to the autonomic nerves to the bladder.

FIGS. 6a, 6b and 6c show front, side and perspective views of a reinforcing implant 800 for use with embodiments of the invention. The implant has a generally Y-shaped form with a tongue 801 and a body 802. In the embodiment shown, body 802 has first and second arms 802a, 802b which contact the anterior and posterior vaginal walls respectively. The arms join the tongue at implant apex 803. However, reinforcing implants having only one arm (e.g. posterior or anterior) joining the tongue at the implant apex, or having a significantly shortened arm (e.g. with one arm extending just beyond the apex), may also be contemplated. In a preferred embodiment, the implant includes one or more frangible regions 804 which facilitate re-sizing of the implant by shortening one or both of the arms prior to implantation. This may be useful e.g. when performing a repair of only the anterior vaginal wall, where the posterior arm may be removed by separating part of the implant along the relevant frangible line. The reinforcing implant tongue may also be trimmable e.g. for repair to the vaginal apex.

In one embodiment, the reinforcing implant may be manufactured from a type 1 light-weight mesh made from a monofilamentous material with pore sizes of around 75 microns, although other materials and pore sizes may be suitable.

In cases where the uterus is present the inventive surgical method may be performed concomitantly with a hysterectomy. Alternatively, if the uterus is conserved then the inventive approach may be used to perform a hysteropexy. When performing a hysteropexy the anterior arm of the implant is removed e.g. by tearing along anterior frangible line or trimming with scissors prior to introduction of the implant into the pelvis. The posterior arm of the implant is attached to the supra-vaginal part of the posterior cervix with a monofilament non-absorbable or delayed absorbable suture (e.g. 2/0 Prolene or 2/0 PDS).

Preferably the reinforcing implant is laser cut and has an anti-adhesion barrier on at least one side of the reinforcing implant tongue 801 so that the pelvic peritoneum does not need to be closed over the implant. Various configurations and sizes are contemplated for the reinforcing implant. Preferably, the reinforcing implant tongue 801 is sufficiently long that it is able to rest along at least a portion of the curvature of the sacral hollow when in placed situ for tissue in-growth. Thus, the tongue may be e.g. 9 cm long or preferably about 13 cm long or as long as 20 or 25 cm. In one embodiment, the tongue is trimmable to accommodate the anatomy of the woman being treated. In one embodiment, the tongue is approximately 4.5 cm wide at its base, gradually tapering to approximately 1 cm width at the tip although this too may vary. The anterior arm 802a may have dimensions of approximately 5 cm length and 4.5 cm width. The posterior arm 802b may have dimensions of approximately 10 cm length and 4.5 cm width. It is to be understood, however, that the invention is not to be limited to the dimensions suggested herein. They are merely provided as a guide.

In a preferred embodiment, the anterior arm has an anti-adhesion barrier facing anteriorly when in situ and in the same orientation as the tongue. Meanwhile, the posterior arm may include an anti-adhesion barrier facing posteriorly when in situ. Thus, both anterior and posterior arms, when enhanced with anti-adhesion properties, have the anti-adhesion barrier facing away from the vagina.

The reinforcing implant 800 is introduced into the abdominal cavity and correctly orientated in the pelvis so that the anti-adhesion barrier on reinforcing implant tongue 801 is facing anterior and the apex 803 of the implant is directed cranially. The anterior and posterior leaves 802a, 802b of the implant are placed onto the upper anterior and posterior vaginal walls respectively as illustrated in FIG. 2. With the splinting appliance in the vaginal canal, anterior and posterior arms of the implant are tacked to the upper anterior and posterior vaginal walls respectively. Preferably this is achieved with a single absorbable suture 215 placed laparoscopically. This is facilitated by the splinting appliance which provides a substantially rigid or semi-rigid support surface enabling the surgeon to tack through the tissue of the vaginal wall. The sutures may be e.g. 2/0 Vicryl or 2/0 Monocryl although other attachment means known in the art may be suitable. Alternatively, the anterior and/or posterior arms of the reinforcing implant may be tacked to the anterior and posterior vaginal walls by a bio-compatible tissue glue or adhesive material.

Once the implant arms have been tacked to the upper vagina, the sacral promontory is identified. The peritoneum over the sacral promontory is grasped and elevated. A first peritoneal opening 210 is formed as a transverse 1.5 to 2 cm incision in the midline to allow entry into the presacral space and the anterior longitudinal ligament 250 is exposed by gentle blunt dissection of the presacral tissues over the sacral promontory. The reinforcing implant tongue 801 may be tucked into the first peritoneal opening. In one embodiment, the tip of the tongue 801 may include a barb, a projection or a widened portion which once inserted into the opening, minimizes the risk of the tongue retracting from the opening while the position of the reinforcing implant is stabilized during tissue in-growth.

In a preferred embodiment, a second peritoneal opening 220 is formed as a transverse 1.5 to 2 cm incision, below first opening, in the midline in the hollow of the sacrum at about the level of the second sacral vertebra. A tunnel is created beneath the peritoneum between the first and second peritoneal openings 210, 220 (e.g.

sacral promontory and mid-sacral peritoneal incisions). This may be facilitated by grasping and elevating the lower part of the first peritoneal opening 210 (i.e. sacral promontory peritoneal incision) with a laparoscopic grasping forceps and introducing a tunneling device into this incision, making a tunnel down to, and then through, the second (i.e. mid-sacral) peritoneal opening 220. The tunneling device may be used to grasp the tip of the reinforcing implant tongue 801 which is then gently drawn back through the tunnel until it comes into abutment with the anterior longitudinal ligament 250 ideally at, and just above, the level of the sacral promontory. Alternatively a second instrument such as forceps may be used to grasp and draw the reinforcing implant tongue 801 through the tunnel. In one embodiment, a single device is used to form the peritoneal openings and the tunnel.

Using this technique, it is possible to position the reinforcing implant 800 so that the reinforcing implant tongue 801 lies in the sacral hollow substantially without tension. The reinforcing implant tongue 801 need not be sutured, anchored, tacked nor stapled to the anterior longitudinal ligament 250. Rather, the vaginal splinting appliance 500 supports the position of the implant 800 during convalescence to allow for fixation of the reinforcing implant tongue 801 onto the sacral promontory and upper sacrum by a process of tissue in-growth into the interstices of the implant. Tissue in-growth may be stimulated by providing growth agents in the vicinity of the reinforcing implant, or onto those surfaces of the reinforcing implant where tissue in-growth is encouraged. Preferably the period of convalescence is approximately 25 days although a period of a few more or a few less days may be required depending on the individual.

At the completion of surgery the pelvis is carefully inspected to ensure adequate haemostasis is present. The vaginal elevator device 600 is withdrawn from the vaginal splinting appliance 500 which remains in situ, secured by sutures 205 as shown in FIG. 2. The sigmoid colon is then gently placed over the reinforcing implant as it lies in the sacral hollow. Cystoscopy may be performed to exclude any urinary tract injury. Digital rectal examination should be performed to exclude any rectal injury. The pneumoperitoneum is released, the laparoscopic instruments are removed and the port site incisions closed. A urethral catheter may be placed.

The surgical methods, appliances and devices disclosed herein aim to simplify procedures for repairing vaginal wall, vaginal vault and/or uterus prolapse so that the surgery may be more readily performed by an increased number of surgeons. By obviating the need for sutures to attach the reinforcing implant to tissue in the sacral promontory, more surgeons will be able to perform this surgery, thereby benefiting larger numbers of women. Use of the vaginal splinting appliance along with the vaginal elevator device and reinforcing implant with an anti-adhesion barrier during the procedure make surgery quicker to perform than other forms of prolapse repair, and with the potential to reduce complications associated with current methods.

Importantly, the inventive methods enable the reinforcing implant to be placed without sutures or other anchoring means in the sacral peritoneum enabling tensionless positioning of the implant. This has the potential to reduce problems associated with tensioned mesh as can arise in traditional therapies. Tensionless implant placement should result in reduced scar tissue formation, reduced implant retraction, less postoperative stress urinary incontinence, less pain and less sexual dysfunction. It is believed that reinforcing implants positioned in accordance with the inventive method can achieve excellent functional outcomes when stabilized with a vaginal splinting appliance during initial tissue in-growth.

As a result of tissue in-growth into the implant, the maximal ‘pull-out’ force of the implant is typically reached 25 days after implantation. By supporting the position of the implant with the vaginal splinting appliance for around 25 days after surgery, fixation of the implant to the sacral promontory by sutures, anchors or staples is not required. Resorbable sutures or fixations for tacking the implant arms onto the vaginal walls are required for initial placement but significant numbers of laparoscopically performed sutures are not required, thereby avoiding intricate and time consuming movements that are required in LSC approaches. This potentially makes the inventive methods simpler, quicker and safer to perform when compared to current methods.

Moreover, placement of the splinting appliance in the vaginal canal at the outset (rather than merely during tissue in-growth) can further facilitate the surgery by providing a support for dissection of tissue from the vaginal walls and placement of sutures to attach the repair graft/implant to the vaginal walls. This approach is contraindicated for repairs performed vaginally but it is anticipated that simplifications to the abdominal approach described herein, particularly when performed laparoscopically, will lead to increased uptake of the new approach to prolapse repair.

While the present invention has been described with reference to a particular example, it is to be understood that various modifications may be made to the surgical method without departing from the ambit of the invention. For instance it is contemplated that the method may involve positioning the tongue of the reinforcing implant along alternative structures such as the uterosacral ligaments. Similarly, modifications, alterations and/or additions may be made to the reinforcing implant, the vaginal splinting appliance and/or the vaginal elevator device without departing from the ambit of the present invention as defined in the claims appended hereto.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or groups thereof.

1. A splinting appliance for use in repair of the vaginal wall or uterus, the appliance including:
a body portion shaped to support a substantially normal vaginal apex;
securing means configured to enable removable securing of the splinting appliance within the vaginal canal; and
coupling means for removably coupling the splinting appliance with a vaginal elevator device when inserted in the vaginal canal during surgery, wherein use of the vaginal elevator device coupled with the splinting appliance aids in location of the vaginal apex;
wherein the splinting appliance provides a substantially rigid support which, when in use, facilitates manipulation of tissue at the vaginal wall when approaching through the abdomen.

2. A splinting appliance according to claim 1, wherein the coupling means is configured to permit decoupling of the vaginal elevator device and the splinting appliance with minimal retraction of the splinting appliance within the vaginal canal.

3. (canceled)

4. A splinting appliance according to claim 1, wherein the securing means includes one or more inwardly directable extension arms configured to be removably attached to the vaginal wall, wherein each one or more extension arm has one or more attachment means selected from the group including: a suture eyelet, a hook and a barb, for attachment of the extension arm to the vaginal wall.

5. (canceled)

6. A splinting appliance according to claim 1, wherein the securing means includes two or more extension arms configured to be joined to one another at one or more positions along the respective extension arms.

7. A splinting appliance according to claim 4 wherein the extension arms include portions that are separable from the appliance to adjust the length of the extension arms.

8. A splinting appliance according to claim 1 wherein the body portion includes one or more removable sections adapted to shorten the body portion when removed.

9. (canceled)

10. (canceled)

11. A vaginal elevator device for use with a vaginal splinting appliance, the elevator device comprising:
(a) coupling zone configured for releasable coupling with the vaginal splinting appliance;
(b) a shaft attached to the coupling zone; and
(c) a handle portion on the shaft for gripping by a user.

12. (canceled)

13. A vaginal elevator device according to claim 11, wherein the handle portion and the shaft are oriented at approximately 30 degrees to one another.

14. A vaginal elevator device according to claim 11, wherein the coupling zone includes a protrusion configured to cooperate with a recess formed in the splinting appliance to engage and controllably manipulate the splinting appliance using the vaginal elevator.

15. (canceled)

16. A method for repairing a vaginal wall or uterus damaged by pelvic organ prolapse in a subject, including the steps of:
(a) forming an access opening to provide abdominal access to the vaginal apex or uterus;
(b) locating a splinting appliance in the vaginal canal;
(c) introducing a reinforcing implant, having a body and a tongue, through the access opening and positioning the body of the reinforcing implant over a portion of the vaginal wall or uterus for repair;
(d) with the splinting appliance positioned in the vaginal canal, attaching at least a portion of the body of the reinforcing implant to one or both of the uterus and the vaginal wall;
(e) positioning the tongue of the reinforcing implant anterior to the sacrum; and
(f) securing the splinting appliance in the vaginal canal for a duration sufficient to achieve tissue in-growth into the reinforcing implant;
wherein securing the splinting appliance in the vaginal canal substantially stabilizes placement of the reinforcing implant while tissue in-growth is occurring.

17. (canceled)

18. A method according to claim 16, wherein the splinting appliance is removed when tissue in-growth has occurred, and wherein there is substantially tensionless placement of the reinforcing implant prior to tissue in-growth.

19. (canceled)

20. A method according to claim 16 wherein securing the splinting appliance in the vaginal canal includes removably attaching at least part of the splinting appliance to the vaginal wall to restrict movement of the splinting appliance.

21. A method according to claim 16, including the step of, prior to attaching the body of reinforcing implant to the vaginal wall, coupling a vaginal elevator device with the splinting appliance, and using the vaginal elevator device to elevate the vaginal vault to facilitate location of the vaginal apex.

22. A method according to claim 16, wherein the duration sufficient to achieve tissue in-growth is 20 to 30 days.

23-24. (canceled)

25. A method according to claim 16, wherein the vaginal wall being repaired includes the posterior vaginal wall, and the method includes, with the splinting appliance positioned in the vaginal canal:
(a) positioning a posterior arm of the body of the reinforcing implant onto an upper portion of the posterior vaginal wall; and
(b) attaching the posterior arm to the vaginal wall.

26. A method according to claim 16, wherein the vaginal wall being repaired includes the anterior vaginal wall, and the method includes, with the splinting appliance positioned in the vaginal canal:
(a) positioning an anterior arm of the body of the reinforcing implant onto an upper portion of the anterior vaginal wall; and
(b) attaching the anterior arm to the vaginal wall.

27. A method according to claim 16 including the steps of:
(a) forming an opening in the peritoneum anterior to the sacral promontory; and
(b) locating a portion of the tongue of the reinforcing implant within the opening, wherein the opening is formed by:
(i) creating a first peritoneal opening anterior to the sacral promontory; and
(ii) forming a tunnel from the first peritoneal opening into the pre-sacral space;
and wherein the tunnel receives at least a portion of the tongue of the reinforcing implant in the region of the sacral hollow, and wherein the tongue of the reinforcing implant is drawn through the tunnel until at least a portion of the tongue of the reinforcing implant rests superior to the peritoneal openings and in abutment with the anterior longitudinal ligament.

28. (canceled)

29. A method according to claim 27 wherein forming the opening further includes:
(a) creating a second peritoneal opening above or below the first peritoneal opening; and
(b) extending the tunnel to the second peritoneal opening, between the peritoneum and the sacrum.

30. A method according to claim 27, wherein each peritoneal opening is a transverse incision in the midline allowing entry into the pre-sacral space, and wherein each transverse incision has a length of 1 to 3 cm, preferably a length of 1.5 to 2.5 cm, and more preferably a length of 1.5 to 2 cm.

31-33. (canceled)

34. A method according to claim 16, including the step of, with a splinting appliance positioned in the vaginal canal, dissecting at least a portion of the posterior and/or anterior vaginal wall from surrounding tissue to facilitate positioning at least a portion of the implant body on the vaginal wall, wherein the substantially rigid support provided by the splinting appliance facilitates dissection of tissue from the vaginal wall and attachment of a repair graft to the vaginal wall.

35-42. (canceled)
